SEQUENCE LISTING (9 sequences)

SEQ ID NO: 1 - DNA, 1058 bp, Artificial Sequence
  lox66-P32-cat-lox71 fragment
aaatctaccgttcgtataatgtatgctatacgaagttatgacaatgtcttaggcgttaaggtcgttttagccgatggtcgcgaagttaagtaaggtaccatgcagtttaaattcggtcctcgggatatgataagaatggcttaataaagcggttactttggatttttgtgagcttggactagaaaaaaacttcacaaaatgctatactaggtagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactgtactttcggatcctaaacgcaattgatgattggttcggaaggcacgttaggaatcattaccgaagtaatcgttaaactgttgccgattccgctagggacccataacttcgtataatgtatgctatacgaacggtacagcccgggcatgag

SEQ ID NO: 2 - DNA, 1601 bp, Lactobacillus plantarum
taaaaccaacattatgacgtgtctgggcatattgccgcccaatgttgcctaacccaacgatcattttcataattttatcttctcctattactttgcataccaaaacaggccgaaccggtaatcgacccgattcggcaactctgagtaacgataccacctaagtcgtattggcaccactactcacaccgtgaccgacgcgcccgccagtcaagtgttcaaaagttagcgtttattaagtgcgataagtataccacaaagggcttattgacgcccgccaaagggttttgcggacattgttaataattgtattaaaagcatgctcaatctaacacttattttgcacaaacatggtatactttaaccgtaaaaactaaattttcactacgagaggatgacttattttgtcaagcatgccaaatcatcaaaaagttgtgttagtcggcgacggcgctgttggttctagttacgcttttgccatggcacaacaaggaattgctgaagaatttgtaattgtcgatgttgttaaagatcggacaaagggtgacgcccttgatcttgaagacgcccaagcattcaccgctcccaagaagatttactcaggcgaatattcagattgtaaggacgctgacttagttgttattacagccggtgcgcctcaaaagcctggtgaatcacgtttagacttagttaacaagaatttaaatatcctatcatccattgtcaaaccagttgttgactccggctttgacggcatcttcttagttgctgctaaccctgttgacatcttaacttacgctacttggaaattctcaggtttcccaaaggatcgtgtcattggttcagggacttccttagactcttcacgtttacgcgttgcgttaggcaaacaattcaatgttgatcctcgttccgttgatgcttacatcatgggtgaacacggtgattctgaatttgctgcttactcaactgcaaccatcgggacacgtccagttcgcgatgtcgctaaggaacaaggcgtttctgacgaagatttagccaagttagaagatggtgttcgtaacaaagcttacgacatcatcaacttgaagggtgccacgttctacggtatcgggactgctttaatgcggatttccaaagccattttacgtgatgaaaatgccgttttaccagtaggtgcctacatggacggccaatacggcttaaacgacatttatatcgggactccggctgtgattggtggaactggtttgaaacaaatcatcgaatcaccactttcagctgacgaactcaagaagatgcaagattccgccgcaactttgaaaaaagtgcttaacgacggtttagctgaattagaaaataaataatcatttcatacgattaaatgtatgatgaacgctcgtctatagcagacgggcgtttttttgtttgcttgaggtaccttagcgattcattaaagcgcaacacgcactaaaggctatttttaaaactttcttatcacgattaccggccttgaagtttgcactcatctcacttctgttataaggtgagaatattacgaatatatggaggaccaacttaattatgaaacataaacgtggact

SEQ ID NO: 3 - DNA, 1490 bp, Artificial Sequence
  mutant L-ldh gene
taaaaccaacattatgacgtgtctgggcatattgccgcccaatgttgcctaacccaacgatcattttcataattttatcttctcctattactttgcataccaaaacaggccgaaccggtaatcgacccgattcggcaactctgagtaacgataccacctaagtcgtattggcaccactactcacaccgtgaccgacgcgcccgccagtcaagtgttcaaatctaccgttcgtataatgtatgctatacgaagttatgacaatgtcttaggcgttaaggtcgttttagccgatggtcgcgaagttaagtaaggtaccatgcagtttaaattcggtcctcgggatatgataagaatggcttaataaagcggttactttggatttttgtgagcttggactagaaaaaaacttcacaaaatgctatactaggtagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactgtactttcggatcctaaacgcaattgatgattggttcggaaggcacgttaggaatcattaccgaagtaatcgttaaactgttgccgattccgctagggacccataacttcgtataatgtatgctatacgaacggtacagcccgggcatgagcttgagctctcgtctatagcagacgggcgtttttttgtttgcttgagggtaccttagcgattcattaaagcgcaacacgcactaaaggctatttttaaaactttcttatcacgattaccggccttgaagtttgcactcatctcacttctgttataaggtgagaatattacgaatatatggaggaccaacttaattatgaaacataaacgtggact

SEQ ID NO: 4 - DNA, 20 bp, Artificial Sequence
  UPF primer
tgaacttgtcgcaacctccg

SEQ ID NO: 5 - DNA, 19 bp, Artificial Sequence
  UPR primer
gaacacttgactggcgggc

SEQ ID NO: 6 - DNA, 20 bp, Artificial Sequence
  DOF primer
tcgtctatagcagacgggcg

SEQ ID NO: 7 - DNA, 20 bp, Artificial Sequence
  DOR primer
gccgtactcttgaactgacg

SEQ ID NO: 8 - DNA, 26 bp, Artificial Sequence
  i128 primer
aaatctaccgttcgtataatgtatgc

SEQ ID NO: 9 - DNA, 20 bp, Artificial Sequence
  i129 primer
ctcatgcccgggctgtaccg

(19) United States
(12) Patent Application Publication
(10) Pub. No.: US 2023/0002796 A1
(43) Pub. Date: Jan. 5, 2023

(54) METHOD FOR INDUCING MICROBIAL MUTAGENESIS TO PRODUCE LACTIC ACID

(71) Applicants: CHIANG MAI UNIVERSITY, Chiang Mai (TH); NATIONAL RESEARCH COUNCIL OF THAILAND, Bangkok (TH)

(72) Inventors: Chartchai KHANONGNUCH, Chiang Mai (TH); Kridsada UNBAN, Chiang Mai (TH); Saisamorn LUMYONG, Chiang Mai (TH); Ronachai PRATANAPHON, Chiang Mai (TH); Wasu PATHOM-AREE, Chiang Mai (TH); Wannisa RIEANTRAKOONCHAI, Chiang Mai (TH); Apinun KANPIENGJAI, Chiang Mai (TH)

(73) Assignees: CHIANG MAI UNIVERSITY, Chiang Mai (TH); NATIONAL RESEARCH COUNCIL OF THAILAND, Bangkok (TH)

(21) Appl. No.: 16/631,048

(22) PCT Filed: Jul. 31, 2018
(86) PCT No.: PCT/TH2018/000034

§ 371 Date: Jan. 14, 2020

(30) Foreign Application Priority Data
Aug. 3, 2017 (TH) 1701004360

(51) Int. Cl. C12P 7/56; C12N 1/20; C12N 15/74; C12N 9/04

(52) U.S. Cl. CPC C12P 7/56; C12N 1/205; C12N 15/746; C12N 9/0006; C12R 2001/25

(57) ABSTRACT

Induction mutagenesis in lactic acid bacteria for D(−) lactic acid production from starch was performed and the stable mutant strain of Lactobacillus plantarum improved by the molecular biological technique can be used in production of high optically pure D(−) lactic acid directly from various kinds of starch as a carbon source. Those starch substrates are included cassava starch, corn starch and rice starch, etc. The fermentation product is high optically pure D(−) lactic acid up to 90.0-99.0% which is able to apply in bioplastic and pharmaceutical industries.

### FIELD OF THE INVENTION

Biotechnology related to ′a method for inducing microbial mutagenesis to produce lactic acid_

### BACKGROUND OF THE INVENTION

Lactic acid is now widely used in various industries such as food, pharmaceutical, cosmetics and the bioplastics industries. Lactic acid has two enantiomeric forms (isomer); L(+) lactic acid (L form) and D(−) lactic acid (D form). The most commonly used lactic acid is L(+) lactic acid regarding the report described the toxicity of D(−) lactic acid humans. Although D (−) lactic acid is not widely used in various industries, but it is essential for some industries such as the pharmaceutical and bioplastics industries. Due to the bioplastics industry requires high purity lactic acid to synthesize polylactic acid (PLA) and improve the quality of PLA bioplastic can be improved by using optimal ratio of D(−) lactic acid to L(+) lactic acid. Generally, production of D(−) lactic acid in industrial scale is produced by microbial fermentation and sugar is used as the main raw material for fermentation. Due to the increase of sugar price, the cost for lactic acid production is also increase as well. Finding for other raw materials to replace sugar, such as starch and lignocellulosic materials is necessary regarding their cheap price and abundantly availability. Nevertheless, obtaining the fermentable sugar from those raw materials requires either the chemical or enzymatic hydrolysis process prior the microbial fermentation process which increases the step the industrial production. Such patent documents appear as follows.

European patent document number EP20100733579 has revealed how to produce D(−) lactic acid from sugar by microbial fermentation and the purifying process using ion exchange resin.

U.S. patent application Ser. No. 07/878,541 discloses a method for the production of D(−) lactic acid by Lactobacillus bulgaricus by fermentation from lactose.

South Korean patent documents PCT/KR2013/003501 has revealed how to produce D(−) lactic acid by Lactobacillus paracasei CC02-0095, the mutant strain achieved by the inhibition of L-lactate dehydrogenase gene (L-ldh) expression, i n the fermentation using De Man, Rogosa and Sharpe (MRS) medium with sugar as a carbon source.

U.S. patent application Ser. No. 10/573813 discloses D(−) lactic acid production by mutant strain of Escherichia coli carrying the NADH-dependent D-lactate dehydrogenase (ldhA) gene in chromosomal DNA using glucose as a carbon source.

European patent document number PCT/EP2013/059186 has revealed how to produce D(−) lactic acid by Lactobacillus corynifornis subsp. torquens strain 30 (ATCC25600) using the fermentable sugars obtained by the enzymatic digestion of steam explosion pretreated cellulosic materials as the carbon sources.

Japanese Patent Document No. PCT/JP2013/058193 has revealed the production of D(−) lactic acid by Escherichia coli strain harboring ldhA and glycerol dehydrogenase (dhaD) genes using glycerol as a carbon source.

From the above patent documents, the production of D(−) lactic acid normally used sugar as the main raw material in the fermentation process. Although there are attempts to create a new strain for achieving the strain with higher D(−) lactic acid capability as shown in the South Korean patent documents. PCT/KR2013/003501, U.S. patent application Ser. No. 10/573,813, but the high cost caused from the use of sugar as the main raw material for lactic acid production still being the same.

Therefore, other cheap raw materials are tried to being used as a carbon source for D(−) lactic acid producing microorganisms. The European Patent Documents PCT/EP2013/059186 has revealed how to produce D(−) lactic acid from the lignocellulosic materials. The substrate must be digested either by steamed explosion or enzymatic digestion to get the fermentable sugar for fermentation process. Disadvantages of those processes are high energy consumption and the high cost of enzyme used in enzymatic digestion. According to the Japan Patent Document Number PCT/JP2013/058193, D(−) lactic acid production using glycerol as the main raw material was revealed, however, the glycerol used as the main substrate must be purified to remove impurities before being used as a carbon source for microbial fermentation. Then, the production cost still remains high.

However, there are no patent documents that revealed the production of D(−) lactic acid by lactic acid bacteria using starch as the main raw material without the conversion process of starch to fermentable sugar.

Production of lactic acid by lactic acid bacteria normally uses pyruvate from Embden-Meyerhof-Parnas pathway. Pyruvate is converted to D(−) or L(+) lactic acid by L-lactate dehydrogenase (EC 1.1.1.27) or D-lactate dehydrogenase (EC 1.1.1.28). Most of lactic acid bacteria in lactobacilli group produce mixed D(−) or L(+) form in the varied ratio and the lactate racemase (EC 5.1.2.1) catalyze the conversion either D(−) to L(+) form or L(+) or D(−) form of lactic acid. There is rare information described on lactate racemase in the previous report. It is normally found in lactobacilli group such as Lactobacillus sakei, Lactobacillus curvatus and Lactobacillus paracasei (Goffin et al., 2005).

Even though it has been revealed that a lactic acid bacterium Lactobacillus plantarum S21 isolated from fermented food collected from north Thailand possess the capability to produce lactic acid directly from starch in the ratio of L(+) to D(−) lactic acid of 1:1 due to the function of L-ldh and D-lactate dehydrogenase (D-ldh) genes in controlling the synthesize of L-lactate dehydrogenase and D-lactate dehydrogenase, respectively. This bacterial strain is stored in the Microbial Resources and Enzyme Technology Laboratory, Faculty of Agro-Industry. Chiang Mai University. L. plantarum S21 produces lactic acid up to 9.4 g/l when cultivation in MRS medium using 10 g/l starch as a carbon source, incubated at 37éC for 15 hours without pH control condition (Kanpiengjai et al., 2014).

However, D(−) lactic acid is preferred for use as a substrate for the production of bioplastics and the production of lactic acid from the cheap substrate as starch can increase the value of agricultural product. Even though it has been revealed that L. plantarum S21 can produce lactic acid directly from starch substrate, but it is produced in the mixed form of D(−) and L(+) lactic acid. There is no information or technology that indicates the microbial strain improvement for only D(−) lactic acid production has been developed.

### SUMMARY OF THE INVENTION

This Invention “a method of induced mutagenesis in lactic acid bacteria for production of D(−) lactic acid from starch” is a method of using lactic acid bacteria Lactobacillus plantarum which is firstly mutated by elimination of L-ldh gene using integrative vector Cre-lox-based system and secondly mutation by Transposon (Tn5) for achieving of D(−) lactic acid directly from starch fermentation.

The purpose for the development of induced mutagenesis in lactic acid bacteria to produce D(−) lactic acid from starch to a high optical purity of 90-99% by the mutant strain of lactic acid bacterium, Lactobacillus plantarum. This reduces the process of conversion of starch to fermentable sugar.

### BRIEF DESCRIPTION OF DRAWINGS

FIG. 1. show the steps to prepare Integrative vector pNZUPDO

FIG. 2. show the gene transferring between lox66-P₃₂-cat-lox71 and L-ldh gene by double-crossing process.

FIG. 3. show the mutation steps of the D1 strain by Transposon Tn5

FIG. 4. shows the comparison of lactic acid produced by of L. plantarum S21 and L. plantarum D1X 1 in liquid MRS containing culture 100 grams per liter of starch as a carbon source under the pH control condition at 6.5-7.0

FIG. 5. shows the comparison of total sugar remaining in the culture of L. plantarum S21 and L. plantarum D1X 1 in liquid MRS containing culture 100 grams per liter of starch as a carbon source under the pH control condition at 6.5-7.0

FIG. 6. shows the amylase activity in the culture of L. plantarum S21 and L. plantarum D1X 1 in liquid MRS containing culture 100 grams per liter of starch as a carbon source under the pH control condition at 6.5-7.0.

FIG. 7. show the number of viable cells in the culture of L. plantarum S21 and L. plantarum D1X 1 in liquid MRS containing culture 100 grams per liter of starch as a carbon source under the pH control condition at 6.5-7.0.

### DETAILED DESCRIPTION OF THE PREFERRED EMBODIMENTS

This invention describes the method for inducing microbial mutagenesis to produce lactic acid, is a method of induced mutagenesis by using integrative vector carrying Cre-lox based system incorporated with transposon Tn5 for the purpose to replace L-ldh gene by a fragment lox66-P₃₂-cat-lox71, and the L(+) lactic acid producing-related gene was blocked by the Tn5 components. This resulted in lactic acid bacterium capable of D(−) lactic acid production with high optical purity up to 99.0-99.9% directly from starch.

Integrative vector possess the Cre-lox based system, is a vector capable of exchanging clusters lox66-P₃₂-cat-lox71 of the target genes in specific bacteria. Specifically, the target gene cannot be expressed. The integrative vector used in this invention is pNZ5319 (Lambert et al., 2007).

Transposon Tn5 is a DNA fragment possesses the ability to insert randomly into the target bacterial gene. The gene being inserted by transposons cannot function properly as an example revealed by Goryshin et al. (2000) that successfully demonstrated the function of Transparent Tn5 in Escherichia coli, Salmonella typhimurium and Proteas vulgaris.

Lactic acid bacteria used in this invention are: Lactobacillus plantarum strain S21 which is a lactic acid bacterium capable of lactic acid producing directly from starch, but this strain produced lactic acid in mixed D(−) and L(+) form in the ratio of 1:1 (after this is simply referred to “Strain S21”).

Lactic acid bacterium strain S21 which is the L-ldh gene is replaced by the lox66-P₃₂-cat-lox71 fragment, leads to malfunction of L-ldh gene, but still remain the capability in production of lactic acid in mixed D(−) and L(+) form directly from starch in the ratio of 1:1 (after this is simply referred to ′Strain D1_).

lox66-P₃₂-cat-lox71 fragment is composed of 1,000 nucleotides containing chloramphenicol resistance gene and the nucleotide sequence which is specific to the digestion of crerecombinase. Nucleotide sequence of lox66-P₃₂-cat-lox71 is as follows:

AAATCTACCGTTCGTATAATGTATGCTATACGAAGTTATGACAATGTCTT
AGGCGTTAAGGTCGTTTTAGCCGATGGTCGCGAAGTTAAGTAAGGTACCA
TGCAGTTTAAATTCGGTCCTCGGGATATGATAAGAATGGCTTAATAAAGC
GGTTACTTTGGATTTTTGTGAGCTTGGACTAGAAAAAAACTTCACAAAAT
GCTATACTAGGTAGATAAAAATTTAGGAGGCATATCAAATGAACTTTAAT
AAAATTGATTTAGACAATTGGAAGAGAAAAGAGATATTTAATCATTATTT
GAACCAACAAACGACTTTTAGTATAACCACAGAAATTGATATTAGTGTTT
TATACCGAAACATAAAACAAGAAGGATATAAATTTTACCCTGCATTTATT
TTCTTAGTGACAAGGGTGATAAACTCAAATACAGCTTTTAGAACTGGTTA
CAATAGCGACGGAGAGTTAGGTTATTGGGATAAGTTAGAGCCACTTTATA
CAATTTTTGATGGTGTATCTAAAACATTCTCTGGTATTTGGACTCCTGTA
AAGAATGACTTCAAAGAGTTTTATGATTTATACCTTTCTGATGTAGAGAA
ATATAATGGTTCGGGGAAATTGTTTCCCAAAACACCTATACCTGAAAATG
CTTTTTCTCTTTCTATTATTCCATGGACTTCATTTACTGGGTTTAACTTA
AATATCAATAATAATAGTAATTACCTTCTACCCATTATTACAGCAGGAAA
ATTCATTAATAAAGGTAATTCAATATATTTACCGCTATCTTTACAGGTAC
ATCATTCTGTTTGTGATGGTTATCATGCAGGATTGTTTATGAACTCTATT
CAGGAATTGTCAGATAGGCCTAATGACTGGCTTTTATAATATGAGATAAT
GCCGACTGTACTTTCGGATCCTAAACGCAATTGATGATTGGTTCGGAAGG
CACGTTAGGAATCATTACCGAAGTAATCGTTAAACTGTTGCCGATTCCGC
TAGGGACCCATAACTTCGTATAATGTATGCTATACGAACGGTACAGCCCG
GGCATGAG

Lactic acid bacterium strain D1 which the chromosomal DNA is inserted and caused the loss of L(+)-lactic acid synthetic process, but still retain the capability in D(−) lactic acid production directly from starch with the purity of 99.0-99.9% (after this is simply referred by ‘Strain D1X1”).

The steps of mutagenesis of the strain S21 is as follows.

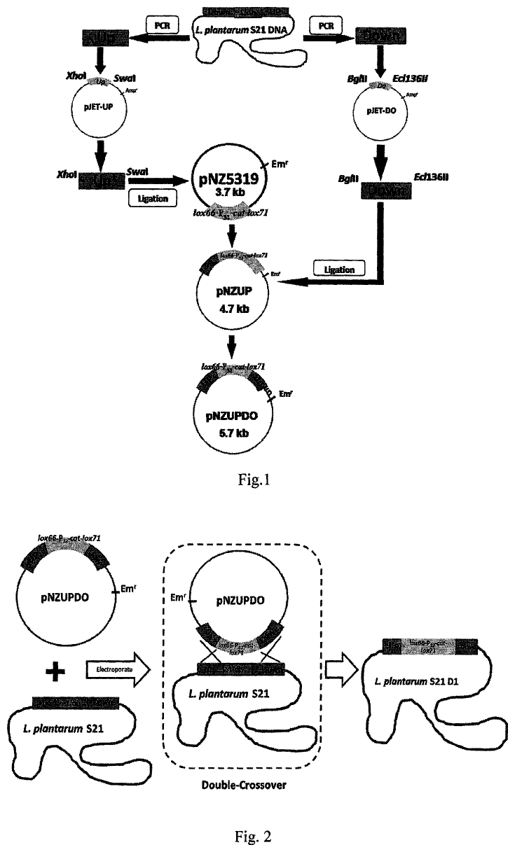

### 1. Preparation of Integrative Vector for Using in Mutagenesis (FIG. 1)

Integrative vector is a vector that has been modified to achieve the capability to exchange a part of its DNA fragment with the target gene in bacterial chromosomal DNA.

Steps of integrative vector preparation are as follows:

1.1 Increase the number of upstream and downstream sequence volumes of L-ldh gene by polymerase chain reaction (PCR) technique using UPF⁻UPR primers and DOF-DOR (Table 1) for increasing the quantities of upstream and downstream region of L-ldh gene.

TABLE 1
Primers used in this invention.

| Primer | 5 [symbol] 3 [symbol] |
|---|---|
| UPF | TGAACTTGTCGCAACCTCCG |
| UPR | GAACACTTGACTGGCGGGC |
| DOF | TCGTCTATAGCAGACGGGCG |
| DOR | GCCGTACTCTTGAACTGACG |
| i128 | AAATCTACCGTTCGTATAATGTATGC |
| i129 | CTCATGCCCGGGCTGTACCG |

[symbol]

indicates data missing or illegible when filed

L-ldh gene is a sequence of nucleotides involved in the production of lactate dehydrogenase which is involved in the production of D(−) lactic acid of the strain S21 is approximately 1,000 nucleotides.

Upstream region of L-ldh gene is the sequence located before the nucleotide gene of the strain S21, approximately 1,000 nucleotides.

Downstream region of L-ldh gene is the sequence located after the nucleotide gene of the strain S21, approximately 1,000 nucleotides.

1.2 Inserted DNA fragment of the upstream region, 1,000 nucleotides, and downstream genes of approximately 1,000 nucleotides of ligand-linked pJET by the enzyme-linked T4 DNA ligase, which produces the pJET-UP and pJET-DO plasmids at this stage.

1.3 Digested pJET-UP with restriction enzymes XhoI and SwaI to separate the L-ldh upstream fragment from the plasmid. The restriction enzyme XhoI and SwaI are specific to the nucleotide sequences 5′-CTCGAG-3 ‘and 5’-ATTTAAAT-3′, respectively. In this step, the DNA fragment approximately 1,000 bp of L-ldh upstream fragment ended with XhoI and SwaI restriction sites will be obtained.

1.4 Digested pJET-DO with restriction enzymes BglII and Ecl136II to separate the L-ldh downstream fragment from the plasmid. The restriction enzyme BglII and Ecl136II are specific to the nucleotide sequences 5′-AGATCT-3 ‘and 5’-GAGCTC-3′, respectively. In this step, the DNA fragment approximately 1,000 bp of L-ldh upstream fragment ended with BglII and Ecl136II restriction sites will be obtained.

1.5 Ligated the 1,000 bp of L-ldh upstream fragment derived from 1.3 into vector pNZ5319 digested with XhoI and SwaI. In this step, the constructed plasmid called pNZUP with approximately 4,700 nucleotides will be achieved.

1.6. Ligated the 1,000 bp of L-ldh downstream fragment derived from 1.4 into vector pNZUP digested with BglII and Ecl136II. In this step, the constructed plasmid called pNZUPDO with approximately 5,700 nucleotides will be achieved.

The DNA fragments containing L-ldh upstream and downstream regions of the strain S21 content were increased. As presented in agarose gel electrophoresis, it was revealed that a 1,000 bp fragment which corresponds to the size of the upstream and downstream regions. Cloning of the L-ldh upstream and downstream DNA fragments in to pJET found that after the upstream and downstream fragments are ligated to the vector and transformed in to E .coli DH5a, the transformant E. coli capable of growth on the medium containing ampicillin was randomly selected and extracted for plasmid vector and cut with BglII. The DNA fragment about 1,000 bp corresponded to the size of upstream and downstream was found. The fragment of 3,000 bp was also corresponded to the size of pJET was also found.

Ligation of the upstream parts of L-ldh gene with XhoI-SwaI digested fragment (about 1,000 bp) into pNZ 5319 digested with XhoI-SwaI (approximately 3,700 bp) and transformed into E. coli DH5a and cultivated on LB agar supplemented with chloramphenicol. The plasmid was extracted and digested with XhoI and BglII found two DNA bands of the upstream region, one is approximately 2,200 bp corresponded to the combination of upstream region and lox66-P₃₂-cat-lox71 and the other band is 2,500 bp which is the size of plasmid vector pNZ5319.

### 2. Mutation of the Strain S21 to Become Strain D1 (FIG. 2)

Four microliters of pNZU PDO was transformed to the competent cell of the strain S21 by the modified method of Posno et al. (1991). It was spread on the MRS agar containing 10ιg/ml chloramphenicol, incubated at 37·C for 48 h. Then, the colonies grown were selected to replicate on MRS agar containing 10ι g/ml erythromycin. The colony capable of growth on MRS agar supplemented with chloramphenicol, but could not grow on the medium containing erythromycin was selected and further cultivated in MRS broth for DNA extraction and used as the template for the polymerase chain reaction to confirm the exchange of lox66-P₃₂-cat-lox71 on pNZUPDO using primers, UPF and i129, i128 and DOR, i128 and i129 (Table 1).

Competent cell is the chemical modified microbial cells to be ready to receive plasmids into cells.

MRS medium is a selective medium used for cultivation of lactic acid bacteria.

A total of 20 colonies capable of growth on the MRS agar supplemented with 10ιg/ml chloramphenicol were obtained. The replica plating for testing the resistance to 10ιg/ml erythromycin was performed. Only 5 colonies including D1, D2, D3, D4 and D5 were not able to grow on the MRS agar containing 10ι g/ml erythromycin. These five colonies were primary confirmed to be the new strains with double crossing over for exchange the fragment of L-ldh gene.

However, the strains D1, D2, D3, D4 and D5 were found to produce mixed L(+) and D(−) lactic acid in a ratio of 1:1 when cultivate in the MRS medium containing starch as a sole carbon source similar to the parental strain, S21. From this result, it can be concluded that even though the L-ldh was successfully deleted from the S21 chromosome but the other biosynthetic pathway responsible for L-lactic acid synthesis (beside the L-ldh gene) still being well functioned. Then the mutant strain L. plantarum D1 was selected for conduct the secondary mutation.

Double crossing over is the DNA fragment exchange process of living organisms.

The nucleotide sequence of the L-ldh gene in the L. plantarum strain S21 is the underlined nucleotide sequence as follows:

TAAAACCAACATTATGACGTGTCTGGGCATATTGCCGCCCAATGTTGCCT
AACCCAACGATCATTTTCATAATTTTATCTTCTCCTATTACTTTGCATAC
CAAAACAGGCCGAACCGGTAATCGACCCGATTCGGCAACTCTGAGTAACG
ATACCACCTAAGTCGTATTGGCACCACTACTCACACCGTGACCGACGCGC
CCGCCAGTCAAGTGTTCAAAAGTTAGCGTTTATTAAGTGCGATAAGTATA
CCACAAAGGGCTTATTGACGCCCGCCAAAGGGTTTTGCGGACATTGTTAA
TAATTGTATTAAAAGCATGCTCAATCTAACACTTATTTTGCACAAACATG
GTATACTTTAACCGTAAAAACTAAATTTTCACTACGAGAGGATGACTTAT
TTTGTCAAGCATGCCAAATCATCAAAAAGTTGTGTTAGTCGGCGACGGCG
CTGTTGGTTCTAGTTACGCTTTTGCCATGGCACAACAAGGAATTGCTGAA
GAATTTGTAATTGTCGATGTTGTTAAAGATCGGACAAAGGGTGACGCCCT
TGATCTTGAAGACGCCCAAGCATTCACCGCTCCCAAGAAGATTTACTCAG
GCGAATATTCAGATTGTAAGGACGCTGACTTAGTTGTTATTACAGCCGGT
GCGCCTCAAAAGCCTGGTGAATCACGTTTAGACTTAGTTAACAAGAATTT
AAATATCCTATCATCCATTGTCAAACCAGTTGTTGACTCCGGCTTTGACG
GCATCTTCTTAGTTGCTGCTAACCCTGTTGACATCTTAACTTACGCTACT
TGGAAATTCTCAGGTTTCCCAAAGGATCGTGTCATTGGTTCAGGGACTTC
CTTAGACTCTTCACGTTTACGCGTTGCGTTAGGCAAACAATTCAATGTTG
ATCCTCGTTCCGTTGATGCTTACATCATGGGTGAACACGGTGATTCTGAA
TTTGCTGCTTACTCAACTGCAACCATCGGGACACGTCCAGTTCGCGATGT
CGCTAAGGAACAAGGCGTTTCTGACGAAGATTTAGCCAAGTTAGAAGATG
GTGTTCGTAACAAAGCTTACGACATCATCAACTTGAAGGGTGCCACGTTC
TACGGTATCGGGACTGCTTTAATGCGGATTTCCAAAGCCATTTTACGTGA
TGAAAATGCCGTTTTACCAGTAGGTGCCTACATGGACGGCCAATACGGCT
TAAACGACATTTATATCGGGACTCCGGCTGTGATTGGTGGAACTGGTTTG
AAACAAATCATCGAATCACCACTTTCAGCTGACGAACTCAAGAAGATGCA
AGATTCCGCCGCAACTTTGAAAAAAGTGCTTAACGACGGTTTAGCTGAAT
TAGAAAATAAATAATCATTTCATACGATTAAATGTATGATGAACGCTCGT
CTATAGCAGACGGGCGTTTTTTTGTTTGCTTGAGGTACCTTAGCGATTCA
TTAAAGCGCAACACGCACTAAAGGCTATTTTTAAAACTTTCTTATCACGA
TTACCGGCCTTGAAGTTTGCACTCATCTCACTTCTGTTATAAGGTGAGAA
TATTACGAATATATGGAGGACCAACTTAATTATGAAACATAAACGTGGAC
T

The nucleotide sequence of the L-ldh gene in the L. plantarum strain D1 which is replaced by lox66-P₃₂-cat-lox71 is underlined.

TAAAACCAACATTATGACGTGTCTGGGCATATTGCCGCCCAATGTTGCCT
AACCCAACGATCATTTTCATAATTTTATCTTCTCCTATTACTTTGCATAC
CAAAACAGGCCGAACCGGTAATCGACCCGATTCGGCAACTCTGAGTAACG
ATACCACCTAAGTCGTATTGGCACCACTACTCACACCGTGACCGACGCGC
CCGCCAGTCAAGTGTTCAAATCTACCGTTCGTATAATGTATGCTATACGA
AGTTATGACAATGTCTTAGGCGTTAAGGTCGTTTTAGCCGATGGTCGCGA
AGTTAAGTAAGGTACCATGCAGTTTAAATTCGGTCCTCGGGATATGATAA
GAATGGCTTAATAAAGCGGTTACTTTGGATTTTTGTGAGCTTGGACTAGA
AAAAAACTTCACAAAATGCTATACTAGGTAGATAAAAATTTAGGAGGCAT
ATCAAATGAACTTTAATAAAATTGATTTAGACAATTGGAAGAGAAAAGAG
ATATTTAATCATTATTTGAACCAACAAACGACTTTTAGTATAACCACAGA
AATTGATATTAGTGTTTTATACCGAAACATAAAACAAGAAGGATATAAAT
TTTACCCTGCATTTATTTTCTTAGTGACAAGGGTGATAAACTCAAATACA
GCTTTTAGAACTGGTTACAATAGCGACGGAGAGTTAGGTTATTGGGATAA
GTTAGAGCCACTTTATACAATTTTTGATGGTGTATCTAAAACATTCTCTG
GTATTTGGACTCCTGTAAAGAATGACTTCAAAGAGTTTTATGATTTATAC
CTTTCTGATGTAGAGAAATATAATGGTTCGGGGAAATTGTTTCCCAAAAC
ACCTATACCTGAAAATGCTTTTTCTCTTTCTATTATTCCATGGACTTCAT
TTACTGGGTTTAACTTAAATATCAATAATAATAGTAATTACCTTCTACCC
ATTATTACAGCAGGAAAATTCATTAATAAAGGTAATTCAATATATTTACC
GCTATCTTTACAGGTACATCATTCTGTTTGTGATGGTTATCATGCAGGAT
TGTTTATGAACTCTATTCAGGAATTGTCAGATAGGCCTAATGACTGGCTT
TTATAATATGAGATAATGCCGACTGTACTTTCGGATCCTAAACGCAATTG
ATGATTGGTTCGGAAGGCACGTTAGGAATCATTACCGAAGTAATCGTTAA
ACTGTTGCCGATTCCGCTAGGGACCCATAACTTCGTATAATGTATGCTAT
ACGAACGGTACAGCCCGGGCATGAGCTTGAGCTCTCGTCTATAGCAGACG
GGCGTTTTTTTGTTTGCTTGAGGGTACCTTAGCGATTCATTAAAGCGCAA
CACGCACTAAAGGCTATTTTTAAAACTTTCTTATCACGATTACCGGCCTT
GAAGTTTGCACTCATCTCACTTCTGTTATAAGGTGAGAATATTACGAATA
TATGGAGGACCAACTTAATTATGAAACATAAACGTGGACT

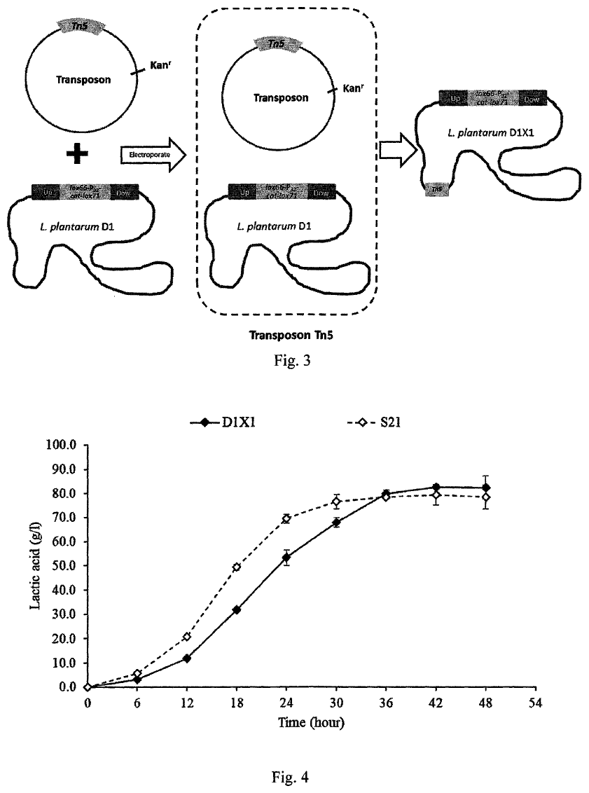

3. Mutation of the Strain D1 to the Strain D1X 1 using Transposable Tn5 (FIG. 3)

Transposon Tn5 can be introduced into the target microorganism by transformation. After the process of transformation into the cells of target microorganisms, Tn5 will randomly insert to bacterial host DNA and cause malfunction or disruption of the gene located at the inserted site and the phenotype will be also interrupted. Transgenic microbes received Tn5 into the cell is able to grow on the medium containing kanamycin. Then, the kanamycin resistant strain can be screened and selected for the target strain showing the desired phenotypes. Regarding to this invention, the target phenotypes of this step is the strain with capability of 50ι g/ml kanamycin and produce only D(−) lactic acid.

Escherichia coli S17-1 harboring the plasmid pSUP 2021 is transferred into LB broth (LB broth is the medium for cultivation of E. coli) containing 50ι g/ml, incubated at 37° C. for 18 hours. The culture was centrifuged at 10,000 rpm for 1 min and the plasmid DNA was extracted by nucleospin plasmid extraction kit (macherey-nagel). The pSUP 2021 obtained was transformed into the competent cell of strain D1 by the modified method of Possno et al. (1991). The transformants was spread on MRS agar supplemented with 50ι g/ml kanamycin, incubated at 37° C. for 48 hours under anaerobic condition. The bacterial colonies capable of growth were selected to cultivate in the MRS broth supplemented with 50ι g/ml kanamycin, incubated at 37° C. for 48 hours. Then, the culture broth was centrifuged at 10,000 rpm 4° C. for 5 min and the supernatant was separated and determined for L(+) lactic acid by the enzymatic method modified from Jehanno et al. (1992). Mixed 10 ml sample with the 50 ml reaction mixture containing NAD 0.65 mM, paraiodonitrotetrazolium 0.59 mM, L-lactate dehydrogenase 4.2 U/ml, diaphoraes 0.108 U/ml, sodium phosphate buffer 0.1 M pH 7.5 and incubated at 37° C. for 30 min. If the sample containing L(+) lactic acid, the color of reaction mixture will change from colorless to pink color. Select colony that is negative in pink color formation to reconfirm for L(+) lactic acid production by enzymatic assay kit (Megazyme, Ireland).

A total of randomly selected 5,600 transgenic bacteria grown on MRS agar containing 10 g/l starch, 50ι g/ml kanamycin and 50ι g/ml chloramphenicol were cultivated in MRS broth and the culture broth produced by each transformants was investigated for L(+) lactic acid production by the enzymatic analytic test kit and it was found that only 3 transformants including D1X1, D1X 2 and D1X 3 showed the negative effect on pink color formation. The transformant D1X 1 was selected as D(−) lactic acid producer with the highest optical purity of 99.0%.

### Example 1: Phenotypic Stability Test of the Strain D1X 1 for D(−) Lactic Acid Production

The phenotypic stability test of lactic acid production for 25 generations, the strain D1X 1 strains showed highly stable to produce D(−) lactic acid directly from starch and retained the optical purity as good as 99.0% optical purity in all 25 generations. It has been demonstrated that lactic acid production by the strain D1X 1 is highly stable.

### Example 2: Growth Comparison Between the Strain D1X 1 and S21

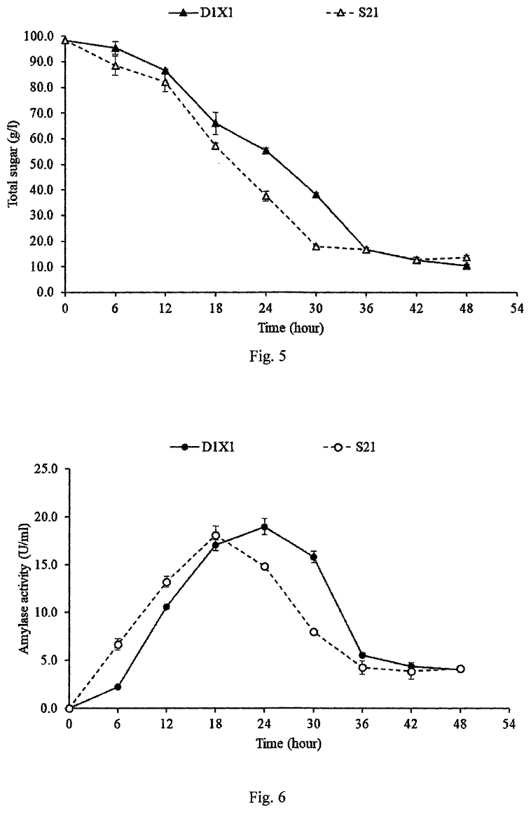

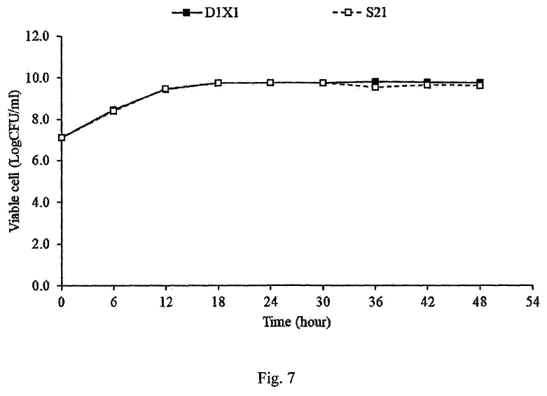

Cultivation of the strain S21 and D1X 1 in the MRS broth using 100 g/l starch as a carbon source, incubated at 37· C for 48 hours. The pH of culture was adjusted to be in range of 6.5-7.0 using 10 M NaOH . Culture broth was sampling with 6 hours interval. It was found that the highest quantity of D(−) lactic acid produced by the original strain and the mutant D1X 1 were 76.5 g/l and 79.8 g/l at 30 and 36 hours, respectively (FIG. 4). The total sugar observed from both cultivations was decreased rapidly at the beginning stage and gradually stabilize after 30 and 36 hours for the original strain and the mutant D1X 1, respectively (FIG. 5). Amylase activity of the strains D1 and D1X 1 were almost similar. The enzyme activity of D1 and D1X 1 strains was gradually increased in the early stages of growth and reached the highest activity at 18.0 and 18.9 U/ml at 18 and 24 hours, respectively (FIG. 6). The number of viable cells of both strains started to be constant at about 9.0 log CFU/ml (FIG. 7). The strain D1X 1 produced lactic acid with the highest quantity, lactic acid yields and productivity of 82.4 ě 1.3 g/l, 0.92 ě 0.05 g/g and 1.71 ě 0.05 g/l/h, which is not different from those value obtained from the D1 strain. However, only the mutant D1X 1 strain produced high optically pure D(−) lactic acid upto 99.0ě0.1% whereas the D1 strain produced mixed D(−) and L(+) lactic acid (Table 2)

TABLE 2
Comparison of the maximum value, yield, productivity and the optical purity
of D(−) lactic acid produced by the parental strain D1 and the mutant D1X1
at 48 hours cultivation in MRS broth using 100 g/l starch as a carbon
source and the pH was adjusted to the range of 6.5-7.0.

| Fermentation parameter/ | The original | The mutant |
|---|---|---|
| Chemical property | strain S21 | strain D1X1 |
| Maximum lactic acid (g/l) | 78.5\check{e}0.5 | 82.4\check{e}1.3 |
| Lactic acid yield (g/g substrate) | 0.91\check{e}0.05 | 0.92\check{e}0.05 |
| Lactic acid productivity (g/l/h) | 1.63\check{e}0.01 | 1.71\check{e}0.05 |
| Optical purity of D(−) lactic acid (%) | 50.1\check{e}0.2 | 99.0\check{e}0.1 |

### BEST MODE FOR CARRYING OUT THE INVENTION

As described in the section of complete disclosure.

1. A process of inducing mutation in lactic acid bacterium Lactobacillus plantarum
to produce D(−) lactic acid, comprising the steps of:
a. Prepare the integrative vector by increasing the quantity of L-ldh gene upstream and downstream fragments using the following primers,
Upstream 5′ → 3′
TGAACTTGTCGCAACCTCCG
and
GAACACTTGACTGGCGGGC
Downstream 5′ → 3′
TCGTCTATAGCAGACGGGCG
and
GCCGTACTCTTGAACTGACG
b. Induces the replacement of lactic acid bacterial L-ldh gene by 1ox66-P₃₂-cat-lox71 fragment by transformation of integrative vector into the competent cell of strain S21, and
c. Replaces the chromosome of lactic acid bacterial strain obtained from the B step with transposon Tn5 for achieving the mutant strain of lactic acid bacteria capable of D(−) lactic acid production from starch substrate.

2. A mutant strain of lactic acid bacterium Lactobacillus plantarum D1X1, wherein the L-ldh gene is replaced by lox66-P₃₂-cat-lox71 fragment and the chromosomal DNA region responsible for lactate racemase is replaced by Tn5 transposon.
